(19) United States
(12) Patent Application Publication
(10) Pub. No.: US 2012/0220852 A1
(43) Pub. Date: Aug. 30, 2012

(54) PATIENT SUPPORT MOTION CONTROL APPARATUS

(75) Inventors: David Bentham, Lexington, MA (US); Alex Wee Kar Tan, Medford, MA (US); Richard Anthony Merhar, Mentor, OH (US); Nabi Abraham Cohn, Chardon, OH (US); Robert Michael Popilock, Hudson, OH (US)

(73) Assignee: KONINKLIJKE PHILIPS ELECTRONICS N.V., EINDHOVEN (NL)

(21) Appl. No.: 13/503,977

(22) PCT Filed: Oct. 14, 2010
(86) PCT No.: PCT/IB2010/054668

§ 371 Date: Apr. 25, 2012

Related U.S. Application Data
(60) Provisional application No. 61/262,264, filed on Nov. 18, 2009.
(60) Provisional application No. 61/264,355, filed on Nov. 25, 2009.

(51) Int. Cl. A61B 6/04

(52) U.S. Cl. 600/407

(57) ABSTRACT

An imaging system (100) includes a stationary gantry (102) having a front side (106) and an examination region (110). The imaging system further includes a patient support (120) configured to position an object or subject thereon in the examination region (110). The imaging system further includes patient support motion controls (122) affixed to the stationary gantry (102) and including a multi-position single control member (202) that controls horizontal, vertical, and diagonal motion of the patient support (120) in and out of the examination region (110).

The following generally relates to a control apparatus for controlling movement of a patient support of an imaging system and finds particular application to computed tomography (CT). However, it also amenable to other imaging systems which use a moveable patient support to move a patient in and out of an examination region before, during and/or after scanning the patient.

Imaging systems such as computed tomography (CT), positron emission tomography (PET), etc. scanners have included an electronically controlled moveable patient support for moving a patient in connection with an examination region of the scanner before, during, and/or after scanning. The movement of the patient support is controllable via commands or instructions automatically or manually (e.g., invoked by user input) sent from an operator console of the imaging system to the patient support. Patient support motion controls have also been incorporated into the cover of the scanner. These controls provide a technician in the examination room the capability of pre-scan loading, positioning, and post-scan unloading a patient from the examination region while in the examination room.

With one such system, the patient support controls include four (4) individual physical buttons respectively for up, down, in, and out patient support motion. With this system, diagonal motion is achieved by simultaneously pressing two (2) buttons (e.g., up and in, down and out, etc.). Another system includes additional buttons for the diagonal motion. These buttons have been grouped together on the scanner cover with other control buttons, leading to a somewhat confusing and complex layout of buttons that often times requires the system operator to focus on the controls to ensure that the correct button is being pressed. Another system employs a touch screen display with graphical virtual buttons rather than physical buttons. These virtual buttons require even more attention as they do not provide any tactile feedback.

With some of the above-noted systems, relatively complex button press schemes are implemented to allow for slow and fast motion. For example, with one system a separate speed change button must be tapped to toggle between fast or slow motion modes prior to pressing the button corresponding to the desired motion direction. Another system includes an additional fifth button the must be pressed and held simultaneously with the desired direction button in order to speed up the motion. As such, this scheme requires two fingers to move the patient in a single direction or three fingers to move the patient diagonally where there is not dedicated diagonal motion button. With yet another system, the speed of the motion automatically increases from slow to fast upon pressing the button for a preset period of time. This may result in under or over shooting the desired position as the operator either anticipates when the change in speed will occur or is unaware of the change in speed.

Some systems have included a foot pedal for speed control and/or patient loading and unloading. Generally, the foot pedal is a separate device that is electrically connected to the imaging system via a cable or the like. Adding such a device further adds to the system and operational complexity. With one interventional system, a video game like joystick control has been used to provide supplemental bi-directional horizontal motion. With this system, the joystick is affixed to the patient support and pivots only along one direction for bi-directional horizontal motion in and out of an examination region. The two-position joystick is generally positioned in connection with the patient support within hands reach of the clinician on the side of the patient support and is designed for the specific purpose of allowing table-side horizontal motion of the patient during an interventional procedure.

The above-noted patient support motion controls, as well as other conventional patient support motion controls, can be somewhat awkward to use and provide less than desirable functionality. For example, with a system that does not provide diagonal motion (e.g., either through dedicated buttons or simultaneous horizontal and vertical motion), the operator has to sequentially move the patient support through vertical and horizontal motions to emulate diagonal motion. As such, the operator may have to alternate between vertical and horizontal motion several times to reach the desired target position. This may require the operator's full attention to the patient support as it moves and reaches various vertical and horizontal limits as defined by the collision envelope and to the controls as the operator has to switch between buttons. This may also increase the amount time it takes to get to position and thus patient exam time, which may decrease patient throughput. In addition, it requires use of multiple control buttons.

Furthermore, touch screen display controls do not provide tactile feedback and thus the technician often has to focus their attention on the touch screen display to ensure that they are actuating the correct control rather than focusing their attention on the patient. With regard to patient support speed control, it can be cumbersome and awkward with control configuration that require multiple fingers to simultaneously press different buttons on a confusing layout or complex button press sequences to switch between speeds. Button press delayed response to switch between speeds can lead to scenarios where the operator has to wait several seconds while the support moves at a slower than desirable speed, followed by a sudden change to a fast speed, at which point an operator not anticipating the change in speed may overshoot the desired target position.

Aspects of the present application address the above-referenced matters and others.

According to one aspect, an imaging system includes a stationary gantry having a front side and an examination region. The imaging system further includes a patient support configured to position an object or subject thereon in the examination region. The imaging system further includes patient support motion controls affixed to the stationary gantry and including a multi-position single control member that controls horizontal, vertical, and diagonal motion of the patient support in and out of the examination region.

According to another aspect, a method includes controlling vertical, horizontal, and diagonal motion of a patient support of an imaging system via a multi-position single control member located on the imaging system.

According to another aspect, patient support motion controls includes a multi-position single control member affixed to the imaging system, wherein the multi-position single control member controls horizontal, vertical, and diagonal motion of the patient support in and out of an examination region of the imaging system.

The invention may take form in various components and arrangements of components, and in various steps and arrangements of steps. The drawings are only for purposes of illustrating the preferred embodiments and are not to be construed as limiting the invention.

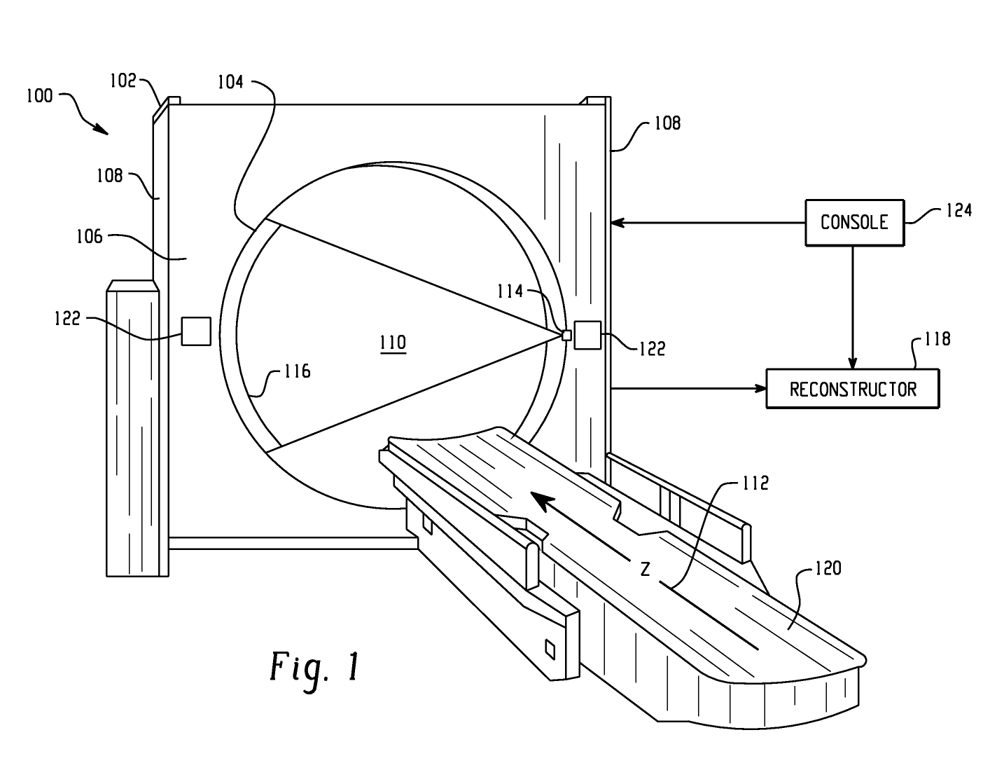

FIG. 1 illustrates an imaging system with patient support motion controls affixed to the imaging system.

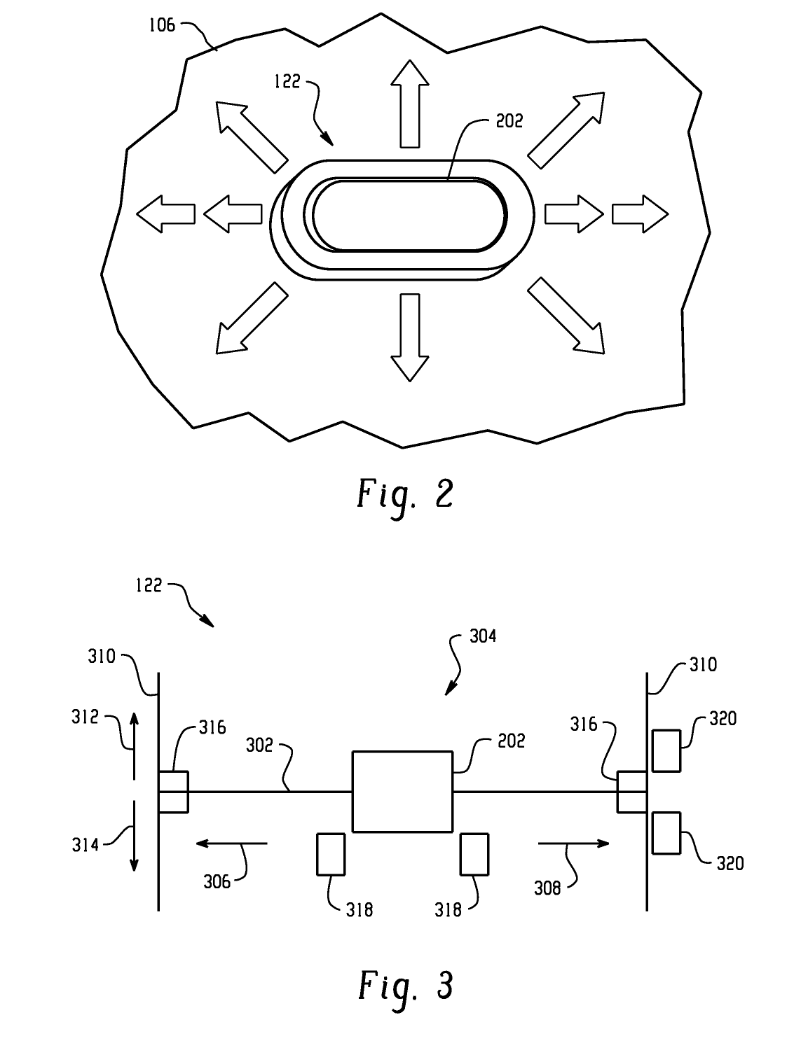

FIG. 2 illustrates an example of a multi-position single member control of the patient support motion controls.

FIG. 3 illustrates example movement of the multi-position single member control to actuate motion.

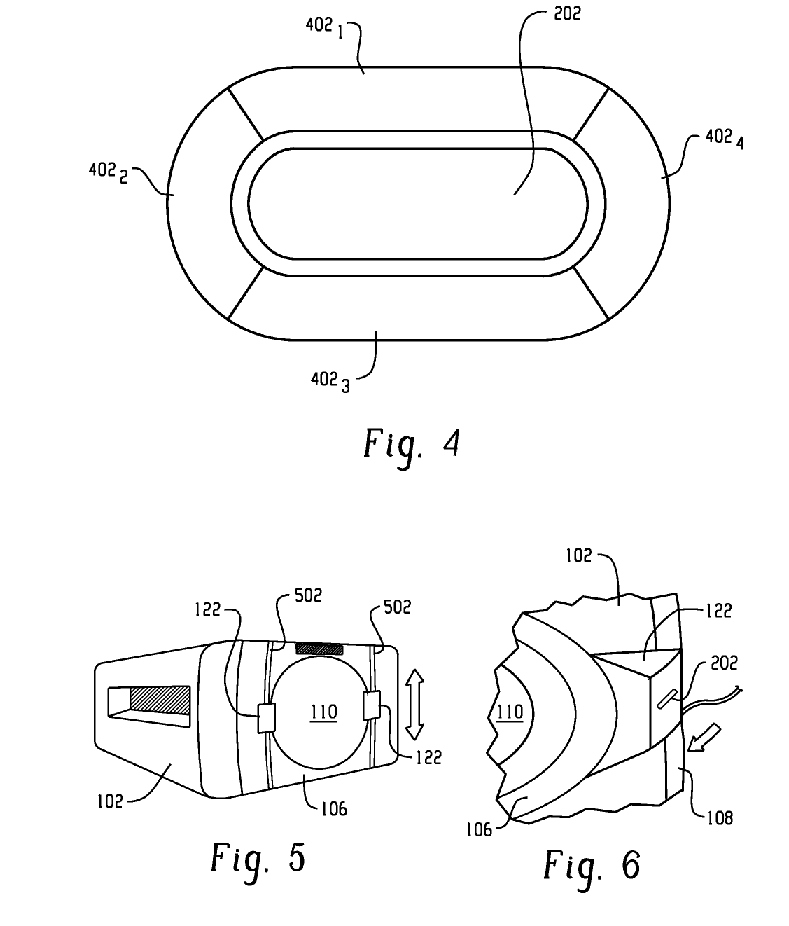

FIG. 4 illustrates example indicators that indicate allowable motion of the patient support motion controls.

FIG. 5 illustrates an embodiment in which the patient support motion controls are moveably affixed to vertical tracks of the front of the imaging system.

FIG. 6 illustrates an embodiment in which the patient support motion controls are affixed to a side of the imaging system.

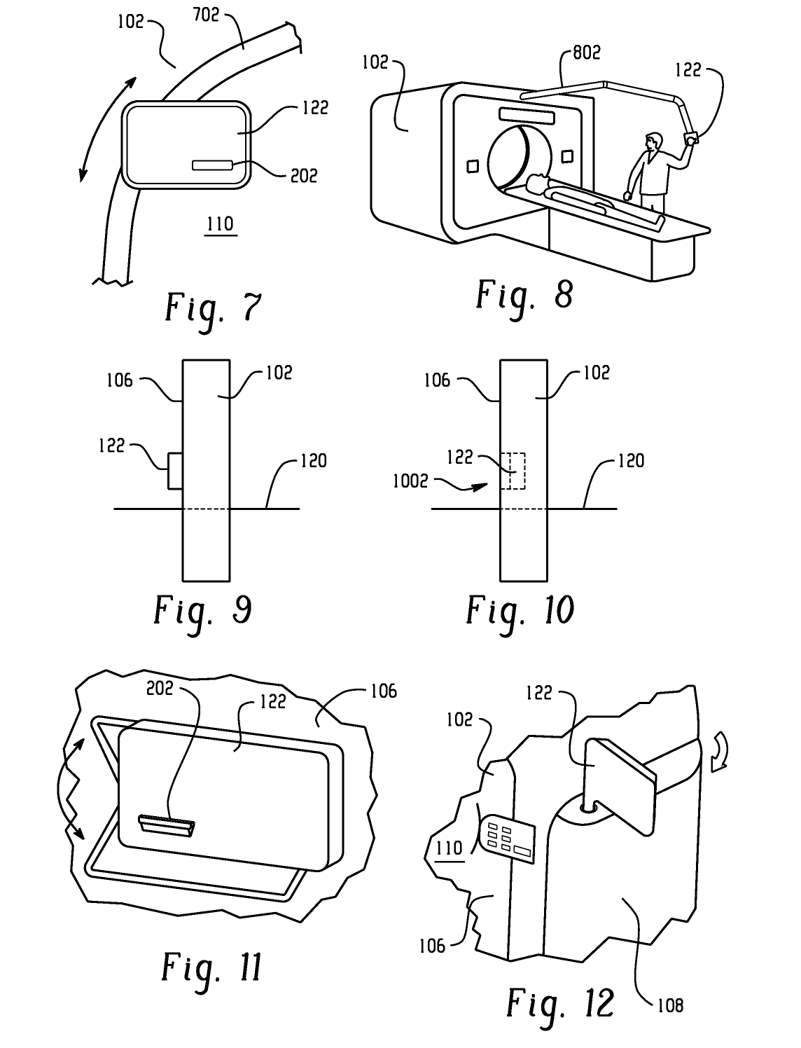

FIG. 7 illustrates an embodiment in which the patient support motion controls are moveably affixed to an annular ring that surrounds the examination region.

FIG. 8 illustrates an embodiment in which the patient support motion controls affixed to a moveable arm of the imaging system.

FIG. 9 illustrates an embodiment in which the patient support motion controls protrude from the face of the imaging system.

FIG. 10 illustrates an embodiment in which the patient support motion controls are located in a recess in the face of the imaging system.

FIG. 11 illustrates an embodiment in which the patient support motion controls are pivotably affixed to the face of the imaging system.

FIG. 12 illustrates an embodiment in which the patient support motion controls are pivotably affixed to the side of the imaging system.

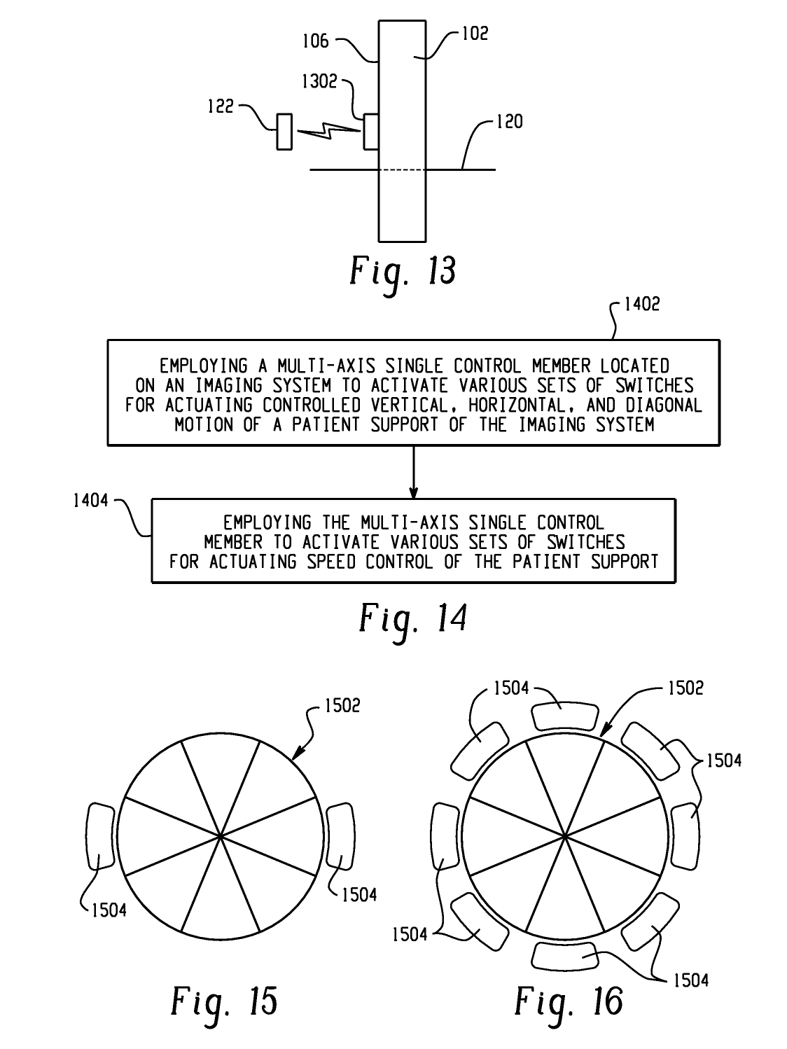

FIG. 13 illustrates an embodiment in which the patient support motion controls remotely communicate with the imaging system.

FIG. 14 illustrates a method of using the patient support motion controls.

FIGS. 15 and 16 illustrate embodiments in which the controls include a rocker switch to control motion direction and at least one other switch to control speed.

The following generally relates to controls for moving a patient support that in connection with an imaging system. For clarity and sake of brevity, the following describes the controls in the context of a computed tomography (CT). However, the controls can similarly be used with other imaging modalities such as magnetic resonance imaging (MRI), positron emission (PET), single photon emission computed tomography (SPECT), ultrasound (US), and/or other imaging modalities.

As described in greater detail below, these controls may include a multi-axis single control member that activates various switches for actuating controlled vertical, horizontal, and diagonal motion, as well as controlling the speed of the motion. Such a member provides intuitive control that allows an operator to move a patient in and out (via a combination of vertical, horizontal, and/or diagonal motion) of an examination region at different speeds from the side of the patient support using a single hand without having to look at the patient support motion controls. It also provides the capability to immediately command different speeds with a single hand. As such, the technician can focus their attention on the patient and move the patient to the desired target location relatively quickly, which may reduce the amount of time a patient has to spend in the examination room and thus increase throughput, improve safety and the accuracy of positioning, etc.

FIG. 1 illustrates an imaging system 100 such as a computed tomography (CT) scanner. However, it is to be appreciated that other imaging modalities are also contemplated herein. The imaging system 100 includes a generally stationary gantry 102 and a rotating gantry 104. The stationary gantry 102 includes at least a front 106 (which is the patient loading and unloading side of the stationary gantry 102 and two sides 108. The rotating gantry 104 is rotatably supported by the stationary gantry 102 and rotates around an examination region 110 about a longitudinal or z-axis 112.

A radiation source 114, such as an x-ray tube, is supported by the rotating gantry 104. The radiation source 114 emits radiation from a focal spot and the radiation traverses the examination region 110. A source collimator includes collimation members that collimate the radiation to form a generally cone, wedge, fan or other shaped radiation beam. A two-dimensional radiation sensitive detector array 116 subtends an angular arc opposite the radiation source 114 across the examination region 110. The detector array 116 includes a plurality of rows of detectors that extend along the z-axis 112 direction. The detector array 116 detects radiation traversing the examination region 110 and generates projection data indicative thereof.

A reconstructor 118 reconstructs the projection data and generates three-dimensional (3D) volumetric image data indicative thereof. The reconstructor 118 may employ a conventional filtered-backprojection reconstruction algorithm, a cone beam reconstruction algorithm, an iterative reconstruction algorithm, and/or other reconstruction algorithm.

A patient support 120, such as a couch, supports an object or subject such as a human patient in the examination region 110. The patient support 120 is configured for vertical (y-axis), horizontal (z-axis), and diagonal (combination of y and z-axis) motion with respect to the examination region 110 before, during and after a scan.

Patient support motion controls 122 control the motion of the patient support 120. In the illustrated embodiment, the patient support motion controls 122 are located on the front side 106 of the stationary gantry 102 and includes two sets of patient support motion controls 122, one on each side of the examination region 110. The patient support motion controls 122 can be located at a height readily visible and accessible to a radiology technician or other authorized personnel standing at the side of the patient support 120. Other embodiments include more or less sets of patient support motion controls 122 located at similar and/or different locations.

A general-purpose computing system or computer serves as an operator console 124. A processor of the console 124 executes computer readable instructions on the console 124, which allows an operator to control operation of the system 100 such as moving the patient support 120, initiating scanning, etc.

FIG. 2 illustrates an example of the patient support motion controls 122. The illustrated patient support motion controls 122 includes a multi-position single control member 202 that is configured to actuate alternatively one or more of a plurality of sets of switches such as two, four, eight, ten, sixteen, etc. sets of switches. The illustrated multi-position single control member 202 is configured to actuate ten (10) sets of switches, including up, down, diagonal in, diagonal out, slow in, slow out, fast in, and/or fast out switches.

In this embodiment, the multi-position single control member 202 includes a slide switch that slides along one or more channels (not visible) to activate the ten different sets of switches to actuate a particular direction of motion and/or speed (e.g., one non-speed, multiple discrete speeds, a variable speed, etc.) of the motion. For reference, direction and speed arrows are included in FIG. 2, with the number of direction arrows corresponding to speed. However, these direction arrows are not physically part of the patient support motion controls 122. Operation is described using these arrows.

Sliding the multi-position single control member 202 in an up direction actuates a set of switches that causes the patient support 120 to move vertically in the up direction at a vertical up speed. Sliding the multi-position single control member 202 in a down direction actuates a set of switches that causes the patient support 120 to move vertically in the down direction. Sliding the multi-position single control member 202 in one of the four (4) diagonal directions actuates a corresponding set of switches that causes the patient support 120 to move diagonally in the selected diagonal direction.

Sliding the multi-position single control member 202 to the left to a first position actuates a set of switches that causes the patient support 120 to move either into or out of examination region 110 at a first speed. Sliding the multi-position single control member 202 further to the left to a second position actuates a set of switches that causes the patient support 120 to move in the same direction but at a second different speed. Sliding the multi-position single control member 202 to the right to a first position actuates a set of switches that causes the patient support 120 to move in the opposite direction at a first speed. Sliding the multi-position single control member 202 further to the right to a second position actuates a set of switches that causes the patient support 120 to move in the same direction but at a second different speed.

In this embodiment, feedback can be provided to the operator so that the operator knows whether the multi-position single control member 202 is in the first or second position when sliding the member left and right. In one instance, tactile feedback (e.g., vibration from a detent or the like) is provided upon crossing from the first to the second position and/or vice versa. Additionally or alternatively, audible feedback (e.g., a beep, a buzz, a message, etc.) is provided upon crossing from the first to the second position or vice versa. Additionally or alternatively, visual feedback (e.g., a light, toggling of a light, an alpha-numeric message is displayed, etc.) is provided upon crossing from the first to the second position or vice versa

It is to be appreciated that the individual motion can be actuated by mechanical contact switches (e.g., liner, rotary, etc.), magnetic switches (e.g., reed switch), and/or other switches. In addition, for vertical and horizontal motion each direction of motion is commanded by sending a corresponding control signal. For diagonal motion, either the corresponding horizontal and vertical motion signals are concurrently sent or a single diagonal motion signal is sent.

FIG. 3 illustrates a non-limiting example of how the multi-position single control member 202 moves. With this example, the patient support motion controls 118 include a first rail 302 to which the multi-position single control member 202 is moveably coupled via a linear bearing such as a ball bearing, a roller bearing, a fluid bearing, or the like. The multi-position single control member 202 is shown positioned at a home or idle position 304.

The multi-position single control member 202 can be held in the home position via a spring, a detent, a magnet, a latch, and/or otherwise. The multi-position single control member 202 is configured to slide along the rail 302 in first and second directions 306 and 308. Sliding the multi-position single control member 202 in one of the directions 306 or 308 activates a switch that actuates patient support motion into the examination region 110. Sliding the multi-position single control member 202 in the opposite direction activates a switch that actuates patient support motion into the examination region 110.

The first rail 302 is support by two second rails 310. The first and second rails 302 and 310 are perpendicular to each other. The first rail 302 is moveably coupled to the second rails 310 via a linear bearing such as a ball bearing, a roller bearing, a fluid bearing, or the like. The first rail 302 is configured to slide along the second rails 310 in third and fourth directions 312 and 314. Sliding the multi-position single control member 202 in one of the directions 312 or 314 activates a switch that actuates patient support vertical up motion. Sliding the multi-position single control member 202 in the opposite direction activates a switch that actuates patient support vertical down motion.

For diagonal motion, the multi-position single control member 202 is slid through a combination of the above motion.

In the illustrated embodiment, switches 318 provide horizontal directional information, switches 316 are used to transition the horizontal speed between slower and higher speeds, and switches 320 provide vertical directional information. The switches 316-320, including their position and size, are provided for explanatory purposes and are not limiting. As noted above, one or more of the switches 316-320 can be mechanical contact, magnetic, and/or other type of switch. Other approaches for determining desired motion direction and speed are contemplated herein.

FIG. 4 illustrates an embodiment in which the indicators 402 (402₁, 402₂, 402₃, and 402₄,) are employed to indicate enabled patient support motion. For example, the indicators 402 may include lighting components that are turned on or illuminated when motion in a particular direction is enabled. Motion in a particular direction may be enabled, for example, when motion in that direction is within a collision envelope or other soft or mechanical bounds of the system.

The illustrated embodiment includes four (4) lighting components that surround the multi-position single control member 202 and that are turned on when motion in that direction is enabled. Diagonal motion is enabled when a corresponding vertical indicator and a corresponding horizontal indicator are turned on. Suitable lighting component include one or more light emitting diodes (LEDs), lasers, and/or other lighting components. Such lights can illuminate the entirety of a visible area of the indicators 402 and/or surrounding the indicators and/or a subportion thereof, for example, a semi-transparent cover or the like. In another embodiment, the lighting components are part of and/or integrated with the multi-position single control member 202.

Variations and other embodiments are contemplated.

As noted above, the multi-position single control member 202 may include more or less then ten positions. By way of example, in another embodiment the up, down and diagonal directions may also move through different speeds, for twelve positions. In another instance, the left and right (in and out) directions are single or more than two speed positions for an eight or greater than ten position control member.

In another embodiment, the multi-position single control member 202 is implemented as a pivotable switch such as a joystick. With this configuration, the switch can be free floating in that it can move directly from one position to another through all ten positions without following a channel or track back through a central position. In another embodiment, the switch pivoting motion is guided along channels.

In another embodiment, the multi-position single control member 202 may additionally include a rotary switch configured to control aspects of patient support 120 motion (e.g., diagonal motion, speed selector, etc.) and/or to control other system functionality such as gantry tilt, an injector, an insuflator, an EKG monitor, a gantry laser light, an alarm, etc.

In another embodiment, the multi-position single control member 202 can be configured to override various soft limits With this embodiment, once motion stops while the multi-position single control member 202 is being activated, the operator can release the multi-position single control member 202 and then being activating the multi-position single control member 202 again in the same direction. If a soft limit override is available, the patient support 120 will again move in that direction until another limit is reached or the operator stop activating the multi-position single control member 202.

In another embodiment, the speed is controlled based on how hard and/or fast the multi-position single control member 202 is moved in a particular direction.

In another embodiment, the multi-position single control member 202 is programmable for at least one of a motion direction or a speed.

In another embodiment, the motion speeds and range of speeds may be controlled in a configurable manner that allows customization by the product designers and/or the customer. For example, the configuration can define the slow speed in each direction and/or the fast speed in each direction.

In another example, the multi-position single control member 202 may be configured so that the patient support 120 moves at one speed for a defined period of time, for example 0.5 seconds, and then releasing the multi-position single control member 202 will toggle the patient support 120 into a predefined jog mode in which the patient support will move through predetermined distance, for example, 0.5 mm.

Although the direction arrows in FIG. 2 are provided as a frame of reference, in another embodiment, the arrows are lighting components that turn on when the multi-position single control member 202 is moved in one of the allowable directions. For example, all of the lights may be off when the multi-position single control member 202 is in a home or idle position. When the multi-position single control member 202 is moved up, the arrow pointing in the up direction can be turned on. The arrows pointing in the other directions are likewise operated. For directions with multi-speeds such as left and right (in and out), with this example, the number of arrows illuminated may indicate the particular speed.

FIGS. 5-10 show alternative mounting locations for the patient support motion controls 122.

FIG. 5 illustrates an embodiment in which a vertical track 502 is mounted to the front side 106 of the stationary gantry 102 and the patient support motion controls 122 are slidably mounted to the vertical track 502. With this embodiment, the patient support motion controls 122 are configured to slide along the vertical track 502 between a plurality of positions. The patient support motion controls 122 can be held at any particular position via various mechanisms such as a set screw, a latch, a detent, a spring loaded lever, or the like. This allows different technicians of different height to individually place the patient support motion controls 122 for access. The patient support motion controls 122 can be mounted via slide, ball, and/or other bearings to the track 502.

FIG. 6 illustrates an embodiment in which the patient support motion controls 122 are mounted on the side 108 of the stationary gantry 102. With this embodiment, the multi-position single control member 202 can be positioned so that the physical direction of the sliding switch corresponds to the direction of the patient support 120. By way of example, in the above embodiment the multi-position single control member 202 slides left and right to move the patient support 120 into and out of the examination region 110. In this embodiment, the multi-position single control member 202 is positioned so that it slides in the direction of the examination region 110 to move the patient support 120 into the examination region 110 and in the opposite direction away from the examination region 110 to move the patient support 120 out of the examination region 110.

FIG. 7 illustrates an embodiment in which the patient support motion controls 122 are moveably mounted on an annular or ring shaped track 702 that is mounted on the stationary gantry 102 so as to surround the examination region 110. Similar to FIG. 5, in this embodiment, the operator can variously adjust the relative location of the patient support motion controls 122 on the stationary gantry 102 to a desired location.

FIG. 8 illustrates an embodiment in which the system 100 includes an extendable and/or retractable arm 802 configured to physically and electronically support the patient support motion controls 122. The arm 802 may have a first member pivotably attached to the system 100 and a second member pivotably attached to the first member via an elbow as shown. In other embodiment, the arm 802 includes more or less members. In addition, the each member may be pivotably attached as in FIG. 8 or attach with a predetermined more limited range of motion.

FIG. 9 illustrates an embodiment in which the patient support motion control 124 are mounted so that they protrude out of the front 106 of the stationary gantry 102, and FIG. 10 illustrates an embodiment in which the patient support motion controls 122 are mounted in a recess 1002 within the front 106 of the stationary gantry 102. With both of the embodiments, the operator is able to rest their hand on the protrusion and/or recess and/or control member 202. This allows to operator to comfortably rest their hand in a position close to the control 122 when they are near the patient support 120 and either not presently activating the controls 122 or are about to active the control 122.

FIG. 11 illustrates an embodiment in which the patient support motion control 122 are pivotably mounted to the front side 106 of the stationary gantry 102. In the illustrated embodiment, the patient support motion controls 122 pivot up and down with respect to the front 106 of the stationary gantry 102. Additionally or alternatively, the patient support motion controls 122 can be configured to pivot side to side with respect to the front of the stationary gantry 102.

FIG. 12 is similar to the embodiment of FIG. 6 with the exception that the patient support motion controls 122 are pivotably mounted to the side 108 of the stationary gantry 102. In this instance, the controls 122 can be positioned as discussed in FIG. 6 so that moving the control member 202 in a direction towards the examination region causes the patient support 120 to move into the examination region (and vice versa), or as discussed in FIG. 1 so that moving the control member 202 left or right causes the patient support 120 to move into the examination region (and vice versa). The patient support motion controls 122 can be swiveled through these positions.

FIG. 13 illustrates an embodiment in which the patient support motion controls 122 detachably affixes to a docking station 1302 on the stationary gantry 102. In this embodiment, the patient support motion controls 122 and the system 100 include wireless communications interfaces, and the patient support motion controls 122 can wirelessly remotely control the motion of the patient support 120.

FIG. 14 describes a method.

At 1402, an operator operates a multi-axis single control member (e.g., member 202) located on an imaging system to activate various sets of switches for actuating controlled vertical, horizontal, and diagonal motion of a patient support 120 of the imaging system 100.

At 1404, the operator operates the multi-axis single control member 202 to activate various sets of switches for actuating speed control of the patient support 120.

The above may be implemented by way of computer readable instructions, which when executed by a computer processor(s), cause the processor(s) to carry out the described acts. In such a case, the instructions are stored in a computer readable storage medium associated with or otherwise accessible to the relevant computer.

FIGS. 15 and 16 illustrate other embodiment of the controls 118. In FIG. 15, the controls 118 include an eight-position rocker switch 1502 or the like that controls motion direction and at least one switch 1504, such as two switches 1504, that activates a fast (or slow) speed for a corresponding at least one of the motion directions. FIG. 16 is similar to FIG. 15 except that it includes a switch 1504 for each of the motion directions. For one or both of FIG. 15 or 16, the switch(s) 1504 is located adjacent to the corresponding direction of motion on the rocker switch 1502. As such, an operator can use a single digit of a hand to actuate both motion direction and speed. Of course, the operator can use more than one digit and/or another instrument, and/or actuate motion without actuating the fast (or slow) speed switch. In other embodiments, the switch(s) is located elsewhere with respect to the rocker switch 1502.

The invention has been described herein with reference to the various embodiments. Modifications and alterations may occur to others upon reading the description herein. It is intended that the invention be construed as including all such modifications and alterations insofar as they come within the scope of the appended claims or the equivalents thereof.

1. An imaging system, comprising:
a stationary gantry having a front side and an examination region;
a patient support configured to position an object or subject thereon in the examination region; and
patient support motion controls affixed to the stationary gantry and including a multi-position single control member that controls horizontal, vertical, and diagonal motion of the patient support in and out of the examination region.

2. The imaging system of claim 1, wherein the multi-position single control member controls at least two non-zero speeds of the motion of the patient support.

3. The imaging system of claim 1, wherein the multi-position single control member includes a slide switch that slides along a channel to activate a set of switches to actuate a desired motion of the patient support.

4. The imaging system of claim 1, wherein the single control member includes at least one of a pivotable joystick that pivots to actuate a desired motion of the patient support or a rotary switch that rotates to actuate a desired motion of the patient support.

5. The imaging system of claim 1, wherein the single control member includes a rocker switch that controls motion direction and at least one other switch that activates a second speed of motion.

6. The imaging system of claim 1, wherein the single control member moves in eight different directions to control both eight directions of motions and at least one of a non-zero speed, multiple discrete speeds, or a variable speed.

7. The imaging system of claim 1, wherein the patient support motion controls provides feedback indicating allowable motion.

8. The imaging system of claim 1, wherein the patient support motion controls are mounted to the front side of the stationary gantry.

9. The imaging system of claim 8, wherein the patient support motion controls protrudes out of the front side.

10. The imaging system of claim 8, wherein the patient support motion controls is affixed in a recess with the front side.

11. The imaging system of claim 1, further comprising: a vertical track mounted to the front side, wherein the patient support motion controls are slidably affixed to the track and move between at least two different vertical positions.

12. The imaging system of claim 1, further comprising: an annular ring shaped track mounted to the front side and surrounding the examination region, wherein the patient support motion controls are slidably affixed to the track and move between at least two different positions along the track.

13. The imaging system of claim 1, further comprising: a extendable/retractable arm affixed to the stationary gantry, wherein the patient support motion controls are affixed to and move with the extendable/retractable arm.

14. The imaging system of claim 1, wherein the patient support motion controls are pivotably affixed to the front side.

15. The imaging system of claim 1, wherein the patient support motion controls are removeably affixed to the imaging system and include a wireless communications interface for remote wireless communication with the patient support.

16. A method, comprising:
controlling vertical, horizontal, and diagonal motion of a patient support of an imaging system via a multi-position single control member located on the imaging system.

17. The method of claim 16, further comprising:
changing a speed of the motion between at least two different speeds via the multi-position single control member.

18. The method of claim 16, wherein the multi-position single control member includes at least one of a rockers switch, slide switch, rotary switch, or a pivot switch.

19. The method of claim 16, wherein the multi-position single control member is affixed on a front side of the imaging system.

20. Patient support motion controls that control a patient support of an imaging system, comprising:
a multi-position single control member affixed to the imaging system, wherein the multi-position single control member controls horizontal, vertical, and diagonal motion of the patient support in and out of an examination region of the imaging system.

21. The patient support motion controls of claim 20, wherein the multi-position single control member toggles the patient support between at least two non-zero speeds.
